(19) United States
(12) Patent Application Publication
(10) Pub. No.: US 2023/0364393 A1
(43) Pub. Date: Nov. 16, 2023

(54) EXPANDABLE SHEATH AND LINER THEREFOR

(71) Applicant: ABIOMED, Inc., Danvers, MA (US)

(72) Inventors: Mithun Rajaram, Danvers, MA (US); Robert Fishman, Danvers, MA (US); Christopher N. Korkuch, Danvers, MA (US); Charles Perkins, Danvers, MA (US)

(73) Assignee: ABIOMED, Inc., Danvers, MA (US)

(21) Appl. No.: 18/195,617

(22) Filed: May 10, 2023

Related U.S. Application Data
(60) Provisional application No. 63/341,252, filed on May 12, 2022.

(51) Int. Cl. A61M 25/06; A61M 39/24; A61M 25/00

(52) U.S. Cl. CPC A61M 25/0662; A61M 39/24; A61M 25/0045

(57) ABSTRACT

An expandable sheath body for a sheath assembly. The sheath body has a tubular frame having a lumen therein. The frame has interstices therein. The frame is expandable to a larger diameter and contractable to a smaller diameter, and also flexible. The sheath body has an inner liner formed over a surface of the lumen defined by the tubular frame and an outer liner formed over an exterior surface of the frame. The inner liner and an inner layer of a multilayer outer liner may be made of expanded polytetrafluoroethylene. The expanded polytetrafluoroethylene may be infused with silicone oil for increased lubricity.

### CROSS REFERENCE TO RELATED APPLICATION

This application claims priority to and the benefit of U.S. Provisional Application No. 63/341,252 filed on May 12, 2022, the disclosure of which is incorporated by reference herein.

### BACKGROUND

Intracardiac heart pump assemblies can be introduced into the heart either surgically or percutaneously and used to deliver blood from one location in the heart or circulatory system to another location in the heart or circulatory system. For example, when deployed in the heart, an intracardiac pump can pump blood from the left ventricle of the heart into the aorta, or pump blood from the inferior vena cava into the pulmonary artery. Intracardiac pumps can be powered by a motor located outside of the patient's body (and accompanying drive cable) or by an onboard motor located inside the patient's body. Some intracardiac blood pump systems can operate in parallel with the native heart to supplement cardiac output and partially or fully unload components of the heart. Examples of such systems include the IMPELLA® family of devices (Abiomed, Inc., Danvers Mass.).

In one common approach, an intracardiac blood pump is inserted by a catheterization procedure through the femoral artery using an introducer sheath, which may be a peel away introducer sheath. The sheath may alternatively be inserted in other locations such as in the femoral vein or any path for delivery of a pump for supporting either the left or right side of the heart.

The introducer sheath may be inserted into the femoral artery through an arteriotomy to create an insertion path for the pump assembly. A portion of the pump assembly is then advanced through an inner lumen of the introducer sheath and into the artery. The requisite size of the arteriotomy is a matter of intense interest. Accordingly, expandable introducer sheaths have been developed so that a smaller arteriotomy opening is required to accommodate the sheath and the medical device passed therethrough. Accordingly, improvements in expandable introducer sheaths continue to be sought.

### BRIEF SUMMARY

According to some aspects, an expandable introducer sheath includes a liner positioned within the sheath, and the liner may be constructed and arranged to reduce friction as a medical device is passed through the sheath. The reduced friction offers easier passage of the medical device through the sheath, and that ease of passage is leveraged to provide an at least nominally smaller diameter sheath that requires an at least nominally smaller arteriotomy opening through which the sheath is introduced into the patient. “Diameter,” as used herein, is used to describe a distance from one side of the introducer sheath or the medical device to the other side. Diameter is not meant to imply that the introducer sheath or introducer sheath lumen or device cross-sections be precisely circular. In some embodiments, the liner is formed from polytetrafluoroethylene (ePTFE).

Described herein is an introducer sheath assembly having a tubular frame having a lumen therein, wherein the tubular frame is configured to temporarily expand from a first diameter to a second larger diameter when a portion of a medical device having a diameter greater than the first diameter passes through the tubular frame. The introducer sheath assembly also has a liner adjacent to an interior surface of the tubular frame, wherein the liner is formed from expanded polytetrafluoroethylene (ePTFE), and a hub coupled to a proximal end of the tubular frame, the hub including a hemostasis valve.

In one aspect, the ePTFE liner is attached to the interior surface of the tubular frame. In another aspect, a primer is formed between the ePTFE liner and an inner surface of the lumen defined by the tubular frame. In another aspect, the introducer sheath assembly has an outer liner formed on an exterior surface of the tubular frame. The tubular frame may be made of braided Nitinol tube or a laser-cut hypotube.

In one aspect, the outer liner is made of thermoplastic polyurethane or expanded polytetrafluoroethylene. The outer line may be single of multilayer. In one aspect the outer liner has two layers. A first layer may be formed over the exterior surface of the frame, and the first layer may be made of a thermoplastic polyurethane. A second layer of the two layers of the outer liner is formed over the first layer, and the second layer may be made of polytetrafluoroethylene.

Also disclosed herein is an expandable sheath body. The expandable sheath body has a tubular frame having a lumen therein, the frame having interstices, wherein the frame is expandable to a larger diameter and contractable to a smaller diameter, and is also flexible. The expandable sheath body has an inner liner formed over a surface of the lumen defined by the tubular frame and an outer liner formed over an exterior surface of the frame. In one aspect, a primer is formed between the inner liner and the surface of the lumen defined by the tubular frame. In a further aspect the primer is formed between the outer liner and the exterior surface of the frame.

In a further aspect, the frame may be made from either braided Nitinol tube or a laser-cut hypotube. In a further aspect, the inner liner may be made of expanded polytetrafluoroethylene. In a further aspect, the expandable sheath may have an outer liner is made of thermoplastic polyurethane or expanded polytetrafluoroethylene and may be multilayer. If the outer liner of the expandable sheath is multilayer, in one aspect the multilayer outer liner may have two layers, wherein a first layer is formed over the exterior surface of the frame, and wherein the first layer comprises thermoplastic polyurethane. A second layer of the two layers of the outer liner is formed over the first layer, wherein the second layer comprises expanded polytetrafluoroethylene.

### BRIEF DESCRIPTION OF DRAWINGS

FIG. 1 illustrates a sheath assembly.

FIG. 2 illustrates a first sheath body configured to be used in a sheath assembly such as that illustrated in FIG. 1.

FIG. 3 is a cross-sectional view of the sheath body of FIG. 2 taken across the A-A axis.

FIG. 4 is a schematic representation of the sidewall portion A of the sheath body of FIG. 3.

FIG. 5 illustrates a second sheath body configured to be used in a sheath assembly such as that illustrated in FIG. 1.

FIG. 6 is a cross-sectional view of the sheath body of FIG. 5 taken across the B-B axis.

FIG. 7 is a schematic representation of the sidewall portion B of the sheath body of FIG. 6.

FIG. 8 illustrates a third sheath body configured to be used in a sheath assembly such as that illustrated in FIG. 1.

FIG. 9 is a cross-sectional view of the sheath body of FIG. 8 taken across C-C axis.

FIG. 10 is a schematic representation of the sidewall portion C of the sheath body of FIG. 9.

### DETAILED DESCRIPTION

Aspects of the present disclosure are described in detail with reference to the figures wherein like reference numerals identify similar or identical elements. It is to be understood that the disclosed aspects are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

To provide an overall understanding of the systems, method, and devices described herein, certain illustrative embodiments will be described. Although the apparatus and its features described herein are specifically described for use in connection with an intracardiac heart pump system, it will be understood that all the components and other features outlined below may be combined with one another in any suitable manner and may be adapted and applied to other types of medical devices such as electrophysiology study and catheter ablation devices, angioplasty and stenting devices, angiographic catheters, peripherally inserted central catheters, central venous catheters, midline catheters, peripheral catheters, inferior vena cava filters, abdominal aortic aneurysm therapy devices, thrombectomy devices, TAVR delivery systems, cardiac therapy and cardiac assist devices, including balloon pumps, cardiac assist devices implanted using a surgical incision, and any other venous or arterial based introduced catheters and devices.

Since commercially available introducer sheaths are typically not radially expandable, the inner diameter of the introducer sheath must always be large enough to accommodate the largest diameter portion of the pump assembly such as the pump head even if other parts of the pump assembly, such as the catheter, have a significantly smaller diameter. In this example, the introducer requires a relatively large blood vessel opening (e.g., an arteriotomy) to allow passage of the pump catheter through the introducer sheath and into the blood vessel, which may complicate arteriotomy closure at the end of a procedure.

Some medical introducers for applications other than inserting heart pumps have expandable sheath bodies that may expand to accommodate passage of larger-diameter percutaneous devices through smaller diameter sheath lumens into the patient's vasculature. Typically, these introducer sheaths, when inserted, have inner diameters smaller than the outer diameter of the device being introduced. The introducer sheaths expand to allow passage of the device through the introducer sheath and into the vasculature and then may relax back to its smaller diameter state after the larger diameter portion of the device has passed through. In the current state of the industry, these expandable introducers require a distinct expandable feature, e.g., a longitudinal fold or crease or a lumen for injection of a fluid (e.g., saline), to transition from a compressed state to an expanded state. As a result, such commercially available expandable sheaths are typically completely flexible and therefore do not provide any rigidity within their structure, thereby leading to kinking or buckling during insertion or withdrawal of a percutaneous medical device. Although expandable sheaths offer many advantages, improvements in design and performance continue to be sought

The systems, methods, and devices described herein provide an expandable sheath body configured for use in an expandable sheath assembly for the insertion of a medical device (e.g., an intracardiac heart pump) into a blood vessel through a vessel aperture. The inventors have recognized and appreciated that expandable sheath systems may provide numerous benefits compared to conventional non-expandable sheaths. For example, by allowing for temporary expansion during insertion of the medical device, an expandable sheath may allow for a smaller blood vessel aperture size (i.e. arteriotomy size) to deliver the medical device compared to a non-expandable sheath, which must have an inner diameter at least as large as the largest diameter of the medical device to be passed therethrough. However, the inventors have further recognized that as the lumen cross-section of an expandable sheath is reduced (e.g., to a nominal or resting cross-section less than the largest diameter portion of a medical device passed therethrough), passage of the medical device through the introducer sheath during insertion and/or removal of the medical device may become more difficult. Accordingly, the systems, methods, and devices described herein provide expandable sheath systems with reduced insertion and/or removal forces that allow for further reduced sheath diameters and correspondingly smaller blood vessel aperture sizes.

The expandable sheath body described herein may be used in an expandable sheath assembly that includes a dilator assembly. As described, the expandable sheath body having an inner surface and an outer surface, the inner surface defining a lumen that extends between proximal and distal ends of the sheath body. Optionally, the expandable sheath assembly may include, in addition to the expandable sheath body, a hemostasis stylet. Expandable sheath assemblies (including the expandable sheath body, dilator assembly, and optional hemostasis stylet) are especially advantageous over existing expandable sheath assemblies for patients with coronary artery disease (CAD) and peripheral artery disease, presenting with calcification and tortuosity of arteries, making delivery of introducer sheaths and catheters difficult.

Expandable sheath assemblies having the sheath body described herein are easier to insert than traditional assemblies because of their reduced insertion profile, increased flexibility, reduced friction, and reduced risk of kinking under loads. The reduced insertion profile reduces insertion related complications, reduces stretching and load on the vessel opening, and reduces the risk of limb ischemia. The structure of the sheath body described herein provides sufficient axial stiffness such that the sheath body can be advanced in a lumen (e.g. a blood vessel) yet resists buckling, while maintaining bending flexibility and kink resistance, and reduces frictional force to prevent “finger trapping.” Moreover, the structures of the sheath body described herein provides an improvement over existing introducer sheath bodies by having at least one of an inner or an outer liner, or both, with each liner constructed and arranged to reduce the force required to expand the sheath body (compared to the force required to expand a sheath having a coating without any bias) and, in the instance of at least a sheath body having the inner liner described herein, for minimizing insertion and removal forces of the device by reducing friction between the sheath body and the device being inserted therethrough.

The sheath body may expand between different states to accommodate the medical device as it is passed through the sheath. For example, the sheath body may be elongated in a first smaller diameter state (typically using a dilator) for insertion and relaxed into a second larger diameter state once at a desired location to allow the passage of a portion of a medical device through the lumen, the portion of the medical device having a transverse cross-sectional area larger than a transverse cross-sectional area of the lumen in the first state. In different configurations, the sheath body diameter is expanded between a resting state when the sheath is deployed at its desired location, and a larger diameter state when the medical device is passed through the sheath body. After the medical device passes through the sheath, the sheath may recoil or otherwise relax and return to the resting state with the smaller diameter. In any configuration, the expandable sheath assemblies having the sheath body described herein do not require additional elements relative to a standard introducer assembly. That is, the assemblies described herein do not have at least the following: i) no external balloon; ii) no fold in the expandable sheath body; and iii) no second sheath for delivery. This simplifies the use of the expandable sheath assembly (e.g., requiring less steps, taking less time) having the sheath body described herein.

Moreover, the momentary expansion of the sheath body from the elongated state to the relaxed state (or from the relaxed state to the expanded state) reduces the size of the opening, e.g., arteriotomy, required when inserting the sheath into the vasculature of the patient. Minimizing the amount of time that the sheath body is in the expanded state also reduces damage to a vessel wall that might otherwise result if the sheath body is maintained in the expanded state for longer periods. Also, the smaller opening needed to receive the sheath body in the relaxed or collapsed state reduces the risk of thrombotic occlusion of the blood vessel that receives the sheath body.

As stated above, the expandable sheath can deliver the sheath body into the vasculature of a patient at a small profile if it is held in axial tension (drawn down) prior to insertion. This has the following key benefits: i) drawing down to a small insertion profile to minimize insertion related complications (i.e., bleeding, vascular injury, high insertion forces); and ii) maintaining a “soft” sheath body and momentary expansion for interaction at the arteriotomy site to allow for small bore closure and minimized bleeding due to minimized vessel recoil during use. However, it should be appreciated that some sheath configurations according to the present disclosure may not be drawn down prior to insertion into a vessel. For example, a sheath may be inserted and maintained in a vessel in a relaxed state and may subsequently momentarily expand to a larger diameter when a medical device passes through the sheath, as described above.

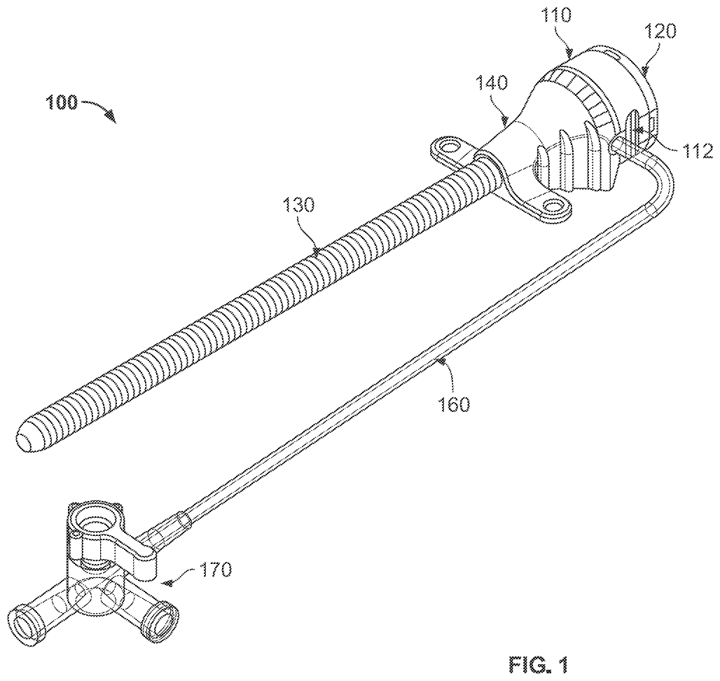

FIG. 1 shows a sheath assembly 100 in accordance with aspects of the presently disclosed technology. The sheath assembly 100 has a hub 110 that locks the sheath in position once inserted. The hub 110 works in concert with the cap 120 to secure the sheath body 130 in position. The hub 110 also has detents 112 (only one of which is visible) to aid in attaching hub 110 to dilator hub 230. The butterfly/suture pad 140 is configured to aid in attaching the sheath assembly 100 to the patient (e.g., by suturing the assembly to the patient). In the present description, the proximal end of the assembly is at the hub/cap end and the distal end of the assembly is at the tip end. Fluid may be introduced into the assembly via sidearm channel 160, and fluid flow into the device may be controlled by stopcock 170. A hemostatic valve (not shown) may also be included within hub 110, the hemostatic valve being configured to prevent blood from leaking outside of the patient during insertion and/or removal of an intracardiac blood pump or other components. Although any suitable hemostatic valve may be employed with the sheath assembly described herein, examples are described and illustrated in U.S. patent application Ser. No. 17/097,582 filed Nov. 13, 2020 which was published as US Patent Publication No. 20210146111 on May 20, 2021, which is incorporated by reference herein. In addition, in some implementations, the hub 110 may include a foam insert (not shown) placed proximal to the hemostatic valve that may be soaked with a lubricant such as silicone so that components will be lubricated as they are inserted through the foam and into the sheath body 130.

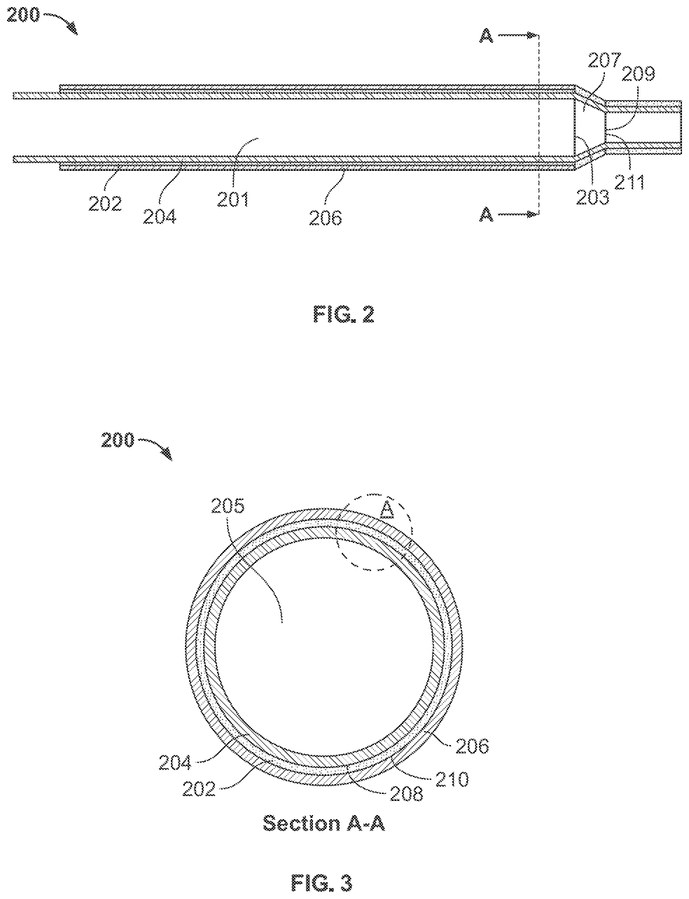

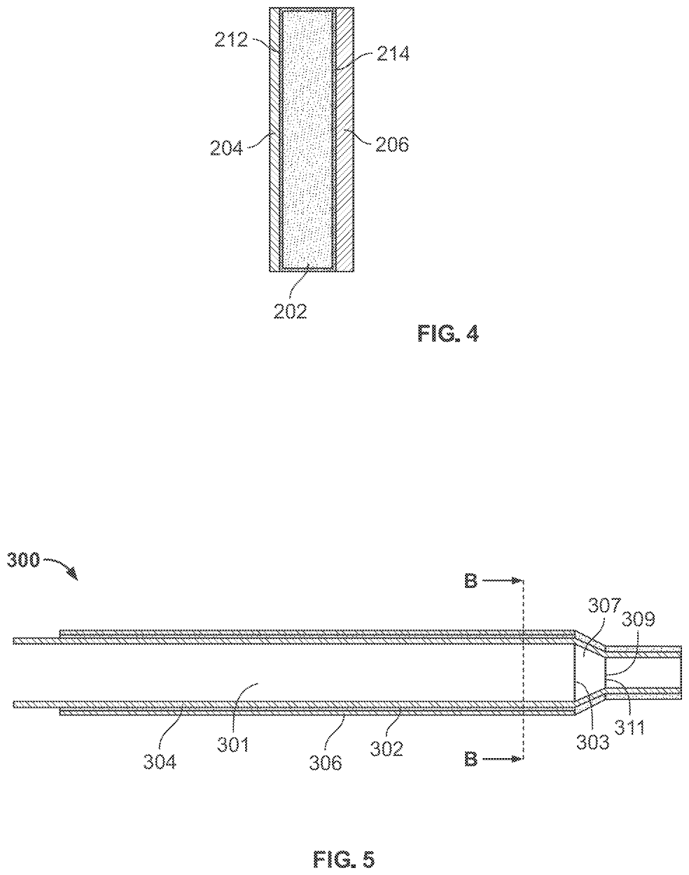

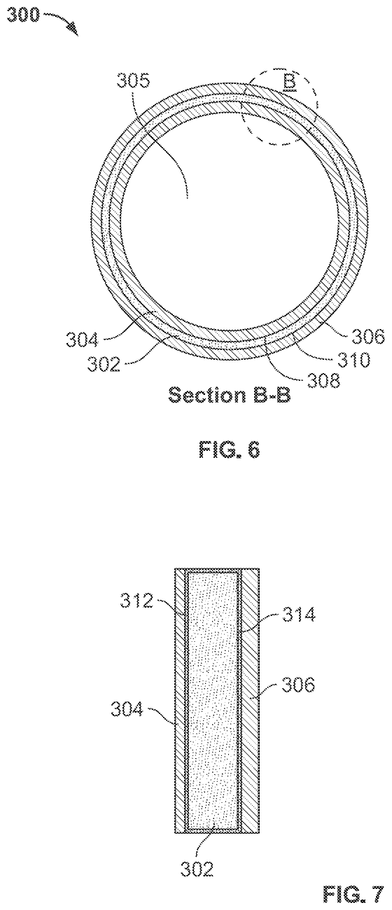

FIGS. 2-10 illustrate various sheath body constructions configured to be used with the sheath assembly 100 in accordance with aspects of the presently disclosed technology. In all aspects, the expandable sheath body 200, 300, 400 includes a multi-layer structure having a circumferential frame 202, 302, 402 interposed between an inner liner 204, 304, 404 and an outer liner 206, 306, 406. The frame 202, 302, 402 may be formed with any suitable material. To provide an expandable frame that is suitably kink resistant, the expandable frame 202, 302, 402 may be a braided material. Alternatively, the frame may be a hypotube, where interstices are introduced by laser cutting. Hypotubes are typically made of metal. In a further aspect the braided material is a metal material having both elasticity and also shape memory properties. One example of a suitable braided material is a braid formed from Nitinol. In some aspects according to the present disclosure, the frame 202, 302, 402 is an expandable braided frame, which is composed of strands of a flexible metal such as Nitinol. The frame 202, 302, 402 may have an expansion mechanism that aids the frame in expanding and/or contracting. For example, strands of the braided frame may be configured with a bias to expand and/or contract from a resting position. According to some implementations, the expansion mechanism permits strands of the braided frame to slide relative to each other when the frame expands and contracts.

Each of the inner liner 204, 304, 404 and outer liner 206, 306, 406 is a low-friction layer that is applied to the frame 202, 302, 402 for mitigating (i.e., reducing) forces required for inserting and removing of the device (e.g., an intracardiac heart pump) by reducing frictional forces caused during the insertion and removal. In addition, the inner liner 204, 304, 404 and outer liner 206, 306, 406 may allow the sheath to expand with a relatively smaller force required compared to the force required to expand the sheath without any liners. As shown in FIGS. 2-10, the inner liner 204, 304, 404 and outer liner 206, 306, 406 are applied (or bonded) to the inner surface 208, 308, 408 of the frame 202, 302, 402 and the outer surface 210, 310, 410 of the frame 202, 302, 402, respectively, using a thermoforming process (also known as lamination). Because the frame is braided in these embodiments, the inner and outer layers may bind to each other through the interstices of the braid. Therefore, the inner and outer liner materials are selected to expand and contract cooperatively with the sheath frame.

As shown in FIGS. 3-4, FIGS. 6-7, and 9-10, the sheath body 200, 300, 400 may further include an inner primer layer 212, 312, 412 interposed between the inner surface 208, 308, 408 of the frame 202, 302, 402 and the inner liner 204, 304, 404, and an outer primer layer 214, 314, 414 interposed between the outer surface 210, 310, 410 of the frame 202, 302, 402 and the outer liner 206, 306, 406. The inner primer layer 212, 312, 412 and outer primer layer 214, 314, 414 are provided to improve adhesion of the inner surface 208, 308, 408 and outer surface 210, 310, 410 of the frame 202, 302, 402 to the inner liner 204, 304, 404 and outer liner 206, 306, 406, respectively.

The inner liner 204, 304, 404 is made of a polymer material. In one aspect, the polymer material is expanded polytetrafluoroethylene (ePTFE). Characteristics of ePTFE provide the inner liner 204, 304, 404 with a smooth, soft, and flexible inner surface for the sheath body 200, 300, 400 that reduces the risk of clot formation and reduces friction and stiction when inserting a device through or removing a device from the expandable sheath. For example, the ePTFE inner liner 204, 304, 404 provides a low coefficient of friction surface in contact with a device being advanced through the sheath body, thereby reducing associated friction forces. In the present disclosure, the outer liner 206, 306, 406 may consist of a single layer or multiple layers of polymer materials (e.g., a multi-layered liner), as will be described in greater detail below.

Suitable primers for the application of ePTFE liners onto a surface are well known and not described in detail herein. ePTFE is hydrophobic, so suitable primers may take this into account. One example of a suitable primer is one that produces amine groups on its surface (e.g., a co-deposition of polyethylenimine (PEI) and polydopamine). Surface treatments such as plasma immersion and oxygen plasma have been used to create a suitable surface for ePTFE deposition. Referring to FIGS. 2-4, in a first aspect, the ePTFE inner liner 204 is applied to the inner surface 208 of the braided frame 202, as stated above. Alternatively, the frame 202 may be formed by patterning a hypotube having a lumen therein. In one aspect, the hypotube is patterned by laser cutting. The pattern that is introduced into the hypotube is provided to control axial expansion, radial expansion, and/or compression of the lumen.

In the present aspect of FIGS. 2-4, the outer liner 206 is also made of a polymer material; the polymer material used to form the outer liner 206 in this aspect may be the same or different from the ePTFE inner liner 204 described above. In the illustrated aspect, the outer liner 206 is made of thermoplastic polyurethane (TPU). Characteristics (or properties) of ePTFE and TPU advantageously provide the smooth, soft, and flexible inner and outer surfaces, respectively, for the sheath body 200 to reduce friction and stiction when inserting a device through or removing a device from the expandable sheath, thereby requiring a relatively smaller force to insert and remove the device (e.g., intracardiac heart pump) than the force required to insert and remove the device from a sheath body without any liners. In addition, the smooth surface of the TPU outer liner 206 advantageously reduces the risk of clots forming on the surface of the expandable sheath body 200. Moreover, in some instances, an inner liner formed from ePTFE may allow for some liquid permeability through the liner, and a liquid impermeable material may be selected for the outer layer. Such a construction may aid in reducing or eliminating undesirable leaking or oozing from the sheath body and may aid in maintaining hemostasis. However, it should be appreciated that the introducer sheaths described herein are not limited to sheaths including a liquid impermeable outer layer. For example, in some embodiments, a sheath including only an ePTFE inner liner may provide a sufficient degree of liquid impermeability to avoid leaking or oozing and maintain hemostasis during use.

In a further aspect, the outer liner may have an outer layer (e.g. TPU) layer that may be partially formed into the microporous structure of the ePTFE.

Referring to FIGS. 5-7, in a second aspect, the ePTFE inner liner 304 is applied to the inner surface 308 of the braided frame 302, as stated above. Like the first aspect described above, alternatively, the frame 302 may be formed by patterning a hypotube having a lumen therein. The hypotube is patterned by laser cutting. The pattern that is introduced into the hypotube is provided to control axial expansion, radial expansion, and compression of the lumen. The inner surface and outer surface of the frame 302 have an inner primer layer 312 and an outer primer layer 314 formed thereon.

In the present aspect, the inner liner 304 and the outer liner 306 are made of the same material. For example, the inner liner 304 and the outer liner 306 are made of ePTFE. Similar to the first aspect, characteristics of ePTFE advantageously provide smooth, soft, and flexible inner and outer surfaces for the sheath body 300 to reduce friction and stiction when inserting a device through or removing a device from the expandable sheath, thereby requiring a relatively smaller force to insert and remove the device (e.g., intracardiac heart pump) than the force required to insert and remove the device from a sheath body without any liners. In addition, the smooth surface of ePTFE outer liner 306 advantageously reduces the risk of clots forming on the surface of the expandable sheath body 300.

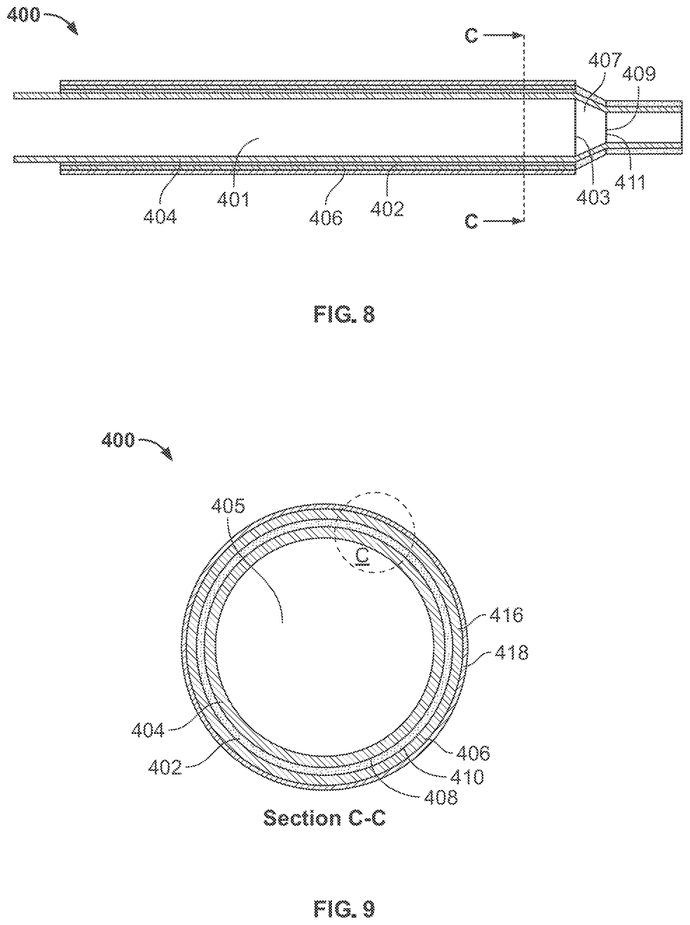

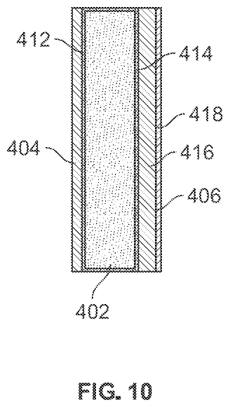

Referring to FIGS. 8-10, in a third aspect, the ePTFE inner liner 404 is applied to the inner surface 408 of the braided frame 402, as stated above. Primer layer 412 is interposed between the inner surface 408 of the frame 402 and the ePTFE inner liner 404. In the present aspect, the outer liner 406 has a multi-layer configuration and thus made of a plurality of polymeric materials. In the illustrated aspect, the outer liner 406 may have an ePTFE layer 416 and a TPU layer 418. As can be seen from FIGS. 9 and 10, the ePTFE layer 416 of the outer liner 406 is adhered to the outer surface 410 of the frame 402 (with the primer layer 414 interposed between the multilayer outer liner 406 and the frame 402). The TPU layer 418 is applied on top of the ePTFE layer 416 to improve the biocompatibility of the ePTFE layer 416. Like the first and second aspects, the ePTFE inner liner 404 and outer multi-layered liner 406 advantageously provide smooth, soft, and flexible inner and outer surfaces, respectively, for the sheath body 400 to reduce friction and stiction when inserting a device through or removing a device from the expandable sheath, thereby requiring a relatively smaller force to insert and remove the device (e.g., intracardiac heart pump) than the force required to insert and remove the device from a sheath body without any liners. In addition, the smooth surface of the multi-layered outer liner 406 advantageously reduces the risk of clots forming on the surface of the expandable sheath body 400.

In the aspects described above, the sheath body 200, 300, 400 may include silicone oil impregnated in the ePTFE inner liner 204, 304, 404 for mitigating or preventing air, water, and/or blood from permeating the ePTFE inner liner 204, 304, 404. The silicone oil also reduces the coefficient of friction of the ePTFE. The coefficient of friction may be reduced by as much a half. The silicone oil is introduced into the ePTFE liner by applying the silicone oil to the surface against which the ePTFE inner liner will be brought into contact. The silicone oil will then wick into the porous ePTFE. When applying the silicone oil to a surface for wicking into the ePTFE, the viscosity of the silicone oil is low enough such that the silicone oil wicks through the ePTFE and is retained by the microporous structure of the hydrophobic ePTFE for providing a resilient lubricious surface. The ability of the silicone oil to be retained by the ePTFE is indirectly proportional to the density and void size of the ePTFE (i.e., for lower density ePTFE (i.e. more porous ePTFE) to exhibit similar silicone oil retainment requires higher viscosity). To achieve a desirable lubricious effect and to obtain a desired retention of silicone oil, the viscosity of silicone oil, for an ePTFE with a density of about 0.4 g/cc, for example, may be 350 cP. Viscosities that are greater than 350 cP do not provide the same lubricious effect. For example, the viscosity of 1000 cP provides only half of the lubricious effect as the viscosity of 350 cP for ePTFEs with densities on the order of those stated above.

In the aspects described above, the inner liner 204, 304, 404 has a tubular shape and extends the entire length of the sheath body 200, 300, 400, as shown in FIGS. 2, 5, and 8. The outer liner 206, 306, 406 also has a tubular shape and extends the length of an elongated portion 201, 301, 401 of the sheath body 200, 300, 400. Thus, the outer liner 206, 306, 406 extends between the proximal end of the sheath body 200, 300, 400 and a division 203, 303, 403, which is a point where the outer surface of the sheath body 200, 300, 400 begins to taper in both inner and outer diameter. As illustrated, beyond the tapered section is a smaller diameter section with a constant inner and outer diameter. As illustrated, the outer liner 206, 306, 406, does not extend over tapered portion or the constant diameter/smaller diameter.

Although the inner diameter and outer diameter of the sheath bodies are largely a matter of design choice, inner diameters of the main sheath body are contemplated to be about 4 mm. About, as used herein, means plus or minus twenty-five percent of the stated value or as one of skill in the art would understand the term in the applicable context. The inner liner 204, 304, 404 and outer liner 206, 306, 406 may have any suitable thickness. In one aspect, the thickness of each of the inner liner 204, 304, 404 and outer liner 206, 306, 406 is approximately 0.05 mm. A lumen 205, 305, 405 of the sheath body 200, 300, 400 may be any suitable size. Preferably, the lumen 205, 305, 405 of the sheath body 200, 300, 400 has an inner diameter of 4 mm. A tapered portion 207, 307, 407 of the sheath body 200, 300, 400 may be any suitable size. Preferably, the length of the tapered portion 207, 307, 407 is between 0.1 mm and 5 mm, and the diameter of the opening 209, 309, 409 at the distal end 211, 311, 411 of the tapered portion 207, 307, 407 is between about 3.7 mm and about 3.9 mm.

Described herein is an introducer sheath assembly that has a tubular frame having a lumen therein, wherein the tubular frame is configured to temporarily expand from a first diameter to a second larger diameter when a portion of a medical device having a diameter greater than the first diameter passes through the tubular frame; a liner adjacent to an interior surface of the tubular frame, wherein the liner is formed from expanded polytetrafluoroethylene (ePTFE); and a hub coupled to a proximal end of the tubular frame, the hub including a hemostasis valve.

In one aspect, the ePTFE liner is attached to the interior surface of the tubular frame. In a further aspect, a primer is formed between the ePTFE liner and an inner surface of the lumen defined by the tubular frame. In another aspect, an outer liner is formed on an exterior surface of the tubular frame.

The frame may be made of a braided Nitinol tube and a laser-cut hypotube. The outer liner may be made of thermoplastic polyurethane, for example expanded polytetrafluoroethylene. In one aspect, the outer liner is multilayer. For example, the outer liner may have two layers, wherein a first layer may be formed over the exterior surface of the frame, and wherein the first layer may be thermoplastic polyurethane. The second layer of the two layers of the outer liner may be formed over the first layer, wherein the second layer comprises expanded polytetrafluoroethylene.

Also described herein is an expandable sheath body that has a tubular frame having a lumen therein, the frame having interstices, wherein the frame is expandable to a larger diameter and contractable to a smaller diameter, and also flexible; an inner liner formed over a surface of the lumen defined by the tubular frame; and an outer liner formed over an exterior surface of the frame.

In one aspect the sheath body may have a primer formed between the inner liner and the surface of the lumen defined by the tubular frame. The primer may be formed between the outer liner and the exterior surface of the frame. The frame may be a braided Nitinol tube or a laser-cut hypotube.

In one aspect of the sheath body, the inner liner may be made of expanded polytetrafluoroethylene. The outer liner may be made of thermoplastic polyurethane, for example expanded polytetrafluoroethylene.

In a further aspect, the outer liner may be multilayer. For example, the outer liner may have two layers, wherein a first layer may be formed over the exterior surface of the frame, and wherein the first layer may be thermoplastic polyurethane. The second layer of the two layers of the outer liner may be formed over the first layer, and the second layer may be formed from expanded polytetrafluoroethylene.

Described herein is an introducer sheath assembly that has a tubular frame having a lumen therein, wherein the tubular frame is configured to temporarily expand from a first diameter to a second larger diameter when a portion of a medical device having a diameter greater than the first diameter passes through the tubular frame. The introducer sheath has a liner adjacent to an interior surface of the tubular frame, wherein the liner is formed from expanded polytetrafluoroethylene (ePTFE) and a hub coupled to a proximal end of the tubular frame, the hub including a hemostasis valve.

In one aspect, the ePTFE liner is attached to the interior surface of the tubular frame. According to the above aspects, a primer may be formed between the ePTFE liner and an inner surface of the lumen defined by the tubular frame. In any of the above aspects, an outer liner is formed on an exterior surface of the tubular frame. In any of the above aspects, the frame is selected from the group consisting of a braided Nitinol tube and a laser-cut hypotube. The frame may be made of Nitinol and the outer liner may be made of thermoplastic polyurethane or expanded polytetrafluoroethylene. In one aspect, the outer liner is multilayer. In another aspect, the outer liner may have two layers, wherein a first layer is formed over the exterior surface of the frame, wherein the first layer comprises thermoplastic polyurethane. In a further aspect, the second layer of the two layers of the outer liner is formed over the first layer, wherein the second layer comprises expanded polytetrafluoroethylene.

Also described herein is an expandable sheath body having a tubular frame having a lumen therein, the frame having interstices, wherein the frame is expandable to a larger diameter and contractable to a smaller diameter, and also flexible. The sheath body may have an inner liner formed over a surface of the lumen defined by the tubular frame and an outer liner formed over an exterior surface of the frame.

In one aspect, a primer is formed between the inner liner and the surface of the lumen defined by the tubular frame. In a further aspect, a primer is formed between the outer liner and the exterior surface of the frame. In the above aspects, the expandable sheath body may have the frame made of a braided Nitinol tube or a laser-cut hypotube. In one aspect, the frame is made of Nitinol. In the above aspects, the expandable sheath body may have the inner liner made of expanded polytetrafluoroethylene. In the above aspects, the outer liner may be made of thermoplastic polyurethane. According to the above aspects, the outer liner may be made of expanded polytetrafluoroethylene. The outer liner may be multilayer. In a further aspect, the outer liner may have two layers, wherein a first layer is formed over the exterior surface of the frame, wherein the first layer comprises thermoplastic polyurethane. In a further aspect, a second layer of the two layers of the outer liner is formed over the first layer, wherein the second layer comprises expanded polytetrafluoroethylene.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

1. An introducer sheath assembly comprising:
a tubular frame having a lumen therein, wherein the tubular frame is configured to temporarily expand from a first diameter to a second larger diameter when a portion of a medical device having a diameter greater than the first diameter passes through the tubular frame;
a liner adjacent to an interior surface of the tubular frame, wherein the liner is formed from expanded polytetrafluoroethylene (ePTFE); and
a hub coupled to a proximal end of the tubular frame, the hub including a hemostasis valve.

2. The introducer sheath assembly of claim 1, wherein the ePTFE liner is attached to the interior surface of the tubular frame.

3. The introducer sheath assembly of claim 1, wherein a primer is formed between the ePTFE liner and an inner surface of the lumen defined by the tubular frame.

4. The introducer sheath assembly of claim 1, wherein an outer liner is formed on an exterior surface of the tubular frame.

5. The introducer sheath assembly of claim 1, wherein the frame is selected from the group consisting of a braided Nitinol tube and a laser-cut hypotube.

6. The introducer sheath assembly of claim 1, wherein the frame is made of Nitinol.

7. The introducer sheath assembly of claim 4, wherein the outer liner is made of thermoplastic polyurethane.

8. The introducer sheath assembly of claim 4, wherein the outer liner is made of expanded polytetrafluoroethylene.

9. The introducer sheath assembly of claim 4, wherein the outer liner is multilayer.

10. The introducer sheath assembly of claim 4, wherein the outer liner comprises two layers, wherein a first layer is formed over the exterior surface of the frame, wherein the first layer comprises thermoplastic polyurethane.

11. The introducer sheath assembly of claim 10, wherein a second layer of the two layers of the outer liner is formed over the first layer, wherein the second layer comprises expanded polytetrafluoroethylene.

12. An expandable sheath body comprising:
a tubular frame having a lumen therein, the frame having interstices, wherein the frame is expandable to a larger diameter and contractable to a smaller diameter, and also flexible;
an inner liner formed over a surface of the lumen defined by the tubular frame;
and an outer liner formed over an exterior surface of the frame.

13. The expandable sheath body of claim 12, wherein a primer is formed between the inner liner and the surface of the lumen defined by the tubular frame.

14. The expandable sheath body of claim 12, wherein a primer is formed between the outer liner and the exterior surface of the frame.

15. The expandable sheath body of claim 12, wherein the frame is selected from the group consisting of a braided Nitinol tube and a laser-cut hypotube.

16. The expandable sheath body of claim 15, wherein the frame is made of Nitinol.

17. The expandable sheath body of claim 12, wherein the inner liner is made of expanded polytetrafluoroethylene.

18. The expandable sheath body of claim 12, wherein the outer liner is made of thermoplastic polyurethane.

19. The expandable sheath body of claim 12, wherein the outer liner is made of expanded polytetrafluoroethylene.

20. The expandable sheath body of claim 12, wherein the outer liner is multilayer.

21. The expandable sheath body of claim 20, wherein the outer liner comprises two layers, wherein a first layer is formed over the exterior surface of the frame, wherein the first layer comprises thermoplastic polyurethane.

22. The expandable sheath body of claim 21, wherein a second layer of the two layers of the outer liner is formed over the first layer, wherein the second layer comprises expanded polytetrafluoroethylene.

23. The expandable sheath of claim 17, wherein the expanded polytetrafluoroethylene is infused with silicone oil.

24. The expandable sheath of any one of claim 23, wherein the expanded polytetrafluoroethylene is infused with silicone oil by wicking the silicone oil from a surface against which a layer of expanded polytetrafluoroethylene is placed.

25. The expandable sheath of claim 23, wherein the expanded polytetrafluoroethylene has a density of about 0.4 g/cc and the silicone oil has a viscosity of about 350 cP.
